(19) United States
(12) Patent Application Publication
(10) Pub. No.: US 2005/0159748 A1
(43) Pub. Date: Jul. 21, 2005

(54) Compression bio-compatible fixation for soft tissue bone fixation

(76) Inventor: Ron Clark, Valparaiso, IN (US)

Correspondence Address: KARL R CANNON, PO BOX 1909, SANDY, UT, 84091, US

(21) Appl. No.: 11/018,856

(22) Filed: Dec. 20, 2004

Related U.S. Application Data
(60) Provisional application No. 60/531,477, filed on Dec. 19, 2003.

(51) Int. Cl. A61F 2/30; A61B 17/58

(52) U.S. Cl. 606/72; 606/60

(57) ABSTRACT

A method of soft tissue graft fixation to bone. A tunnel may be formed in a bone and a bio-compatible material may be placed in the tunnel in a liquid state using an insertion device. The soft tissue grafts may also be placed in the tunnel using the insertion device. The insertion device may serve as an internal mold to form a chamber for receiving a fastener. The liquid bio-compatible material may solidify around the insertion device, and the insertion device may be removed. Final fixation may be accomplished by inserting a fastener in the hardened bio-compatible material to apply a compressive force to compress the graft against the bone.

### CROSS-REFERENCE TO RELATED APPLICATIONS

This application claims the benefit of U.S. Provisional Application No. 60/531,477, filed Dec. 19, 2003, which is hereby incorporated by reference herein in its entirety, including but not limited to those portions that specifically appear hereinafter, the incorporation by reference being made with the following exception: In the event that any portion of the above-referenced provisional application is inconsistent with this application, this application supercedes said above-referenced provisional application.

### STATEMENT REGARDING FEDERALLY SPONSORED RESEARCH OR DEVELOPMENT

Not Applicable.

### BACKGROUND

1. The Field of the Invention

The present disclosure relates generally to ligament and tendon graft attachment devices and methods, and more particularly, but not necessarily entirely, to a ligament fixation system and method utilizing a matrix, such as a compressive bio-compatible material.

2. Description of Related Art

Apparatus and methods for attaching ligaments to bone have been developed and are preexisting in the prior art. Typically, these devices either attach a ligament graft to the exterior of a bone, or within a drill hole bored through the endosteal portion of the bone. The strength of the graft attachment in such systems is usually dependent on the cancellous bone density of the bone.

One of the several known methods for fixing ligaments to bone includes the IntraFix® method. The IntraFix® method of tibial hamstring fixation represents an effective form of tibial fixation for hamstring anterior cruciate ligament (ACL) reconstruction that utilizes an interference screw. The ACL, which runs from the front of the tibia to the back of the femur, helps to stabilize the knee by preventing the tibia from moving too far forward. The demands placed on the knee sometimes exceed its limits and if the ligament is stretched to tightly, it can tear or rupture. This type of injury commonly occurs to athletes when they quickly pivot or change directions, causing the upper leg to be turned outward while the lower leg is turned inward. When the ACL is injured, it can be surgically repaired to restore the knee's stability and function.

The IntraFix® method of ligament fixation utilizes an expansion sheath and a tapered screw. A tunnel is formed in the tibia and the expansion sheath is placed into the tunnel between soft tissue graft strands. Then the screw is inserted into the sheath to compress the graft strands against the tunnel sidewall and thereby fix the graft to the bone. This method has the advantages of an all endosteal technique and the elimination of metallic artifact on imaging studies. Its popularity is tempered by those who would eliminate all foreign, non-biodegradable, material from ACL reconstruction because it uses a polyethylene sleeve as an interface between the strands of the graft and the interference screw. One embodiment of the new technique herein presented serves to resolve this dilemma by utilizing a bio-compatible compound that may be introduced in a liquid state by an insertion device. The bio-compatible compound may serve as a negative mold for an interference screw.

Additional background disclosure is provided in U.S. Pat. No. 6,558,389, which reference is hereby incorporated by reference herein in its entirety.

The prior art is thus characterized by several disadvantages that are addressed by the present disclosure. The present disclosure minimizes, and in some aspects eliminates, the above-mentioned failures, and other problems, by utilizing the methods and structural features described herein.

The features and advantages of the disclosure will be set forth in the description which follows, and in part will be apparent from the description, or may be learned by the practice of the disclosure without undue experimentation. The features and advantages of the disclosure may be realized and obtained by means of the instruments and combinations particularly pointed out in the appended claims.

### BRIEF DESCRIPTION OF THE DRAWINGS

The features and advantages of the disclosure will become apparent from a consideration of the subsequent detailed description presented in connection with the accompanying drawings in which:

FIG. 1 is a perspective view of an insertion device;

FIG. 1A is a side view of the insertion device of FIG. 1;

FIG. 2 is a perspective view of a fastener;

FIG. 2A is a side view of the fastener of FIG. 2;

FIG. 3 is an exploded perspective view of a hex driver, fastener and hardened matrix;

FIG. 4 is side cross-sectional view of a bone having a tunnel with the insertion device inserted;

FIG. 5 is side cross-sectional view of a bone having a tunnel with the insertion device inserted and a liquid matrix being deployed;

FIG. 6 is side cross-sectional view of a bone having a tunnel with the insertion device removed and the matrix in place;

FIG. 7 is side cross-sectional view of a bone having a tunnel with matrix in place and a fastener partially inserted; and

FIG. 8 is side cross-sectional view of a bone having a tunnel with matrix in place and a fastener fully seated.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

The publications and other reference materials referred to herein to describe the background of the disclosure, and to provide additional detail regarding its practice, are hereby incorporated by reference herein in their entireties, with the following exception: In the event that any portion of said reference materials is inconsistent with this application, this application supercedes said reference materials. The reference materials discussed herein are provided solely for their disclosure prior to the filing date of the present application. Nothing herein is to be construed as a suggestion or admission that the inventors are not entitled to antedate such disclosure by virtue of prior disclosure, or to distinguish the present disclosure from the subject matter disclosed in the reference materials.

In describing and claiming the present disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps. Moreover, as used in this specification and the appended claims, the singular forms “a,” “an,” and “the” include plural referents unless the context clearly dictates otherwise.

As used herein, the term “liquid” shall be construed broadly to include substances that have the ability to flow or deform, including substances in a paste or dough-like state.

As used herein, the term “solidify” shall be construed broadly to include situations in which a substance becomes solid, hardened, or strong so as to no longer exist in a liquid state.

As used herein, the term “implant” shall be construed broadly to include tissues, objects or devices imbedded into or on a body, including items, such as tendon or ligament grafts, as well as other naturally occurring or artificial prosthetic devices.

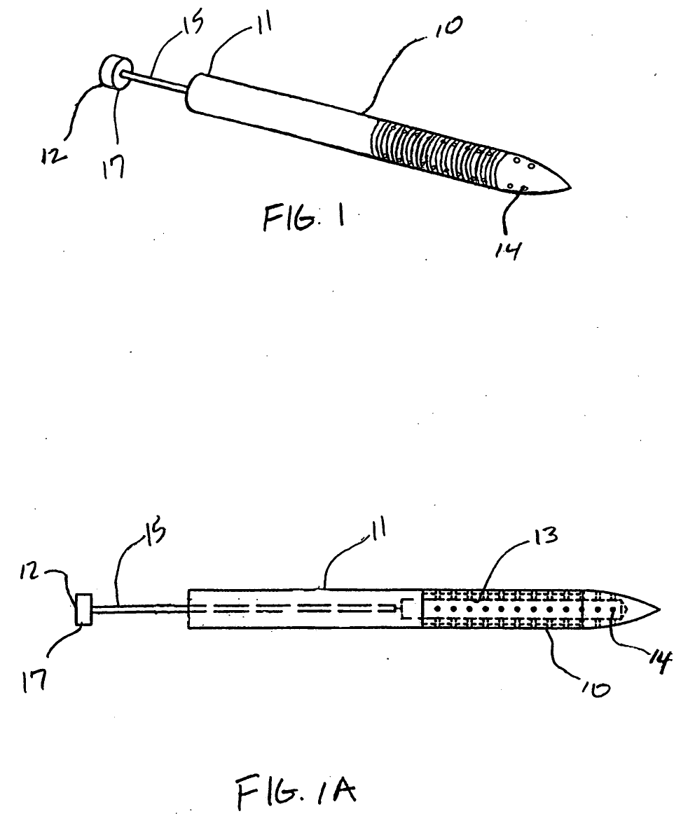

Referring now to FIG. 1, a perspective view of a hollow insertion device or mold 10 is shown. A side view of the insertion device 10 is shown in FIG. 1A. The insertion device 10 may include a housing wall 11 defining a reservoir 13. The insertion device 10 may also include a plunger 12 at least partially receivable within the housing wall 11 for forcing the contents of reservoir 13 out of the insertion device 10. The plunger 12 may include a shaft 15 for extending within the housing wall 11, and a handle 17 for providing a surface area to apply a force to move the shaft 15. It will also be understood that a gasket (not shown) may be disposed on a distal end of the shaft 15, opposite the handle 17, to improve the ability of the plunger 12 to move the contents of the insertion device 10 in a manner known to those skilled in the art.

The housing wall 11 of the insertion device 10 may be fenestrated at 14 to allow passage of a material from within the reservoir 13 to outside of the insertion device 10 upon depression of the handle 17 of the plunger 12. In one embodiment, the fenestrations 14 may be configured in four rows extending along a length of the insertion device 10. The rows may be spaced substantially equidistantly apart around the perimeter of the insertion device 10. This configuration of the fenestrations 14 may facilitate positioning the materials ejected from the insertion device 10 evenly around the perimeter of the insertion device 10. This configuration may also reduce the distance the materials ejected from the insertion device 10 must travel to encompass the insertion device 10. Accordingly, use of the insertion device as a negative mold may be facilitated, as discussed more fully below. It will be understood that the fenestration configuration may vary within the scope of the present disclosure such that any number of openings in various sizes and locations may be provided. For example, one embodiment of the insertion device 10 may include a single opening, whereas other embodiments may have a plurality of openings arranged in any number of rows or any other pattern or configuration.

It will also be appreciated that the insertion device 10 may be formed in various different sizes and configurations within the scope of the present disclosure. Moreover, it will be understood that other embodiments of the insertion device 10 may be formed without a plunger 12, in that other mechanisms may be used to expel the contents of the insertion device 10. For example, the insertion device 10 may be provided with a flexible bulb or bag capable of receiving and expelling a fluid material when compressed.

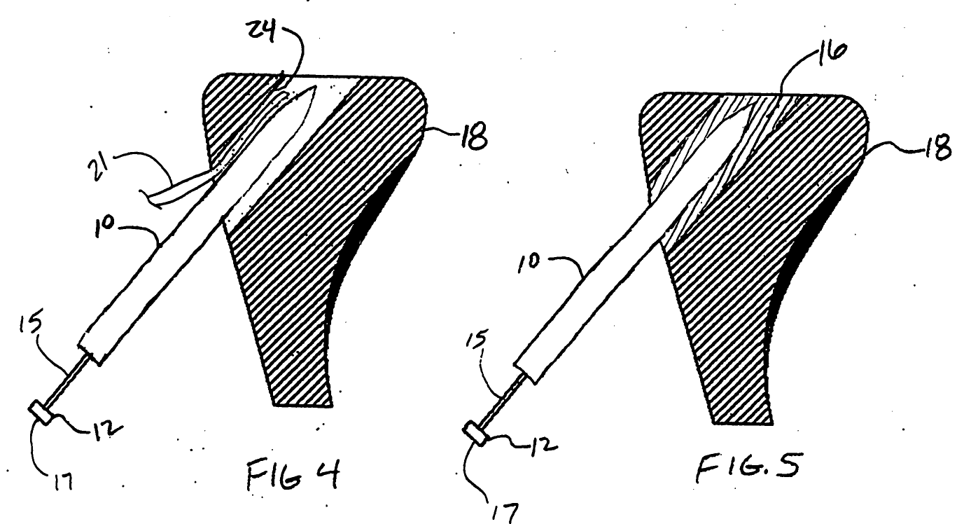

A matrix 16, such as a liquid bio-compatible material, as depicted schematically in FIG. 5, may be placed in the insertion device 10 to be inserted in a bone 18 as described more fully below. The matrix 16 may include a substance that may be formed in a liquid state, including a flowable paste or dough-like state, which may become solid or solidify, such as through a chemical reaction and/or a change in temperature. The material properties of the matrix 16 to be inserted in the bone 18 are likely to be enhanced over time. Initially, materials such as Mimix® calcium-based paste may be recommended. Another exemplary embodiment of the matrix 16 may include a polymer such as a polymethylmethacrylate (PMMA) material. It will be understood that any of a variety of PMMA materials known to those skilled in the art may be used, or other materials forming the matrix 16 may be used within the scope of the present disclosure. In one embodiment of the present disclosure, methylmethacrylate may be polymerized in an exothermic reaction that may result in a doughy substance that may self cure and solidify in a short time. Those skilled in the art will appreciate that the chemical composition of the matrix 16 may be configured in various different embodiments to achieve the desired properties, such as strength, rigidity, durability, and cure time, for example, within the scope of the present disclosure. Moreover, it will be understood that enhanced bio-mechanical performance may be achieved by alterations of set time, density, and adjuvants such as concentrated platelets or growth factors.

Another embodiment of the matrix 16 may include a calcium-phosphate material. For example, a monocalcium phosphate, mono-hydrate, a-tricalcium phosphate, and calcium carbonate may be mixed dry then suspended in a sodium phosphate solution. This process may result in the formation of an injectable paste that may begin to harden in minutes to form a carbonated apatite (dahllite) through a non-exothermic reaction. The calcium-phosphate material may have beneficial characteristics. For example, the calcium-phosphate material may be osteoconductive, have substantial compressive strength, and may be resorbed or converted to host bone over time.

A further embodiment of the matrix 16 may include a material derived from a hydrated mixture which comprises KH₂PO₄, a metal oxide, and a calcium containing compound. For example, one mixture may include 45 percent KH₂PO₄, 45 percent MgO, 10 percent calcium-containing compound such as CaSiO₃ or Ca₁₀(PO₄)₆(OH)₂, and 25 percent by weight water. While the above formulation and weight percentages are described as an example, a range of the constituents also may be utilized. For example, between 40 and 50 percent by weight of the KH₂PO₄ may be utilized, as well as between 35 and 50 percent by weight of the MgO. The ratio of KH₂PO₄ to the MgO may be between 1:0.5 and 1:1, for example. Also, aside from MgO, various other oxide and hydroxide powders may be utilized, including, but not limited to FeO, Al(OH)₃, Fe₂ O₃, Fe₃O₄ and Zr(OH)₄. Additional disclosure regarding the above described material is disclosed in U.S. Pat. No. 6,533,821, which reference is hereby incorporated by reference herein in its entirety.

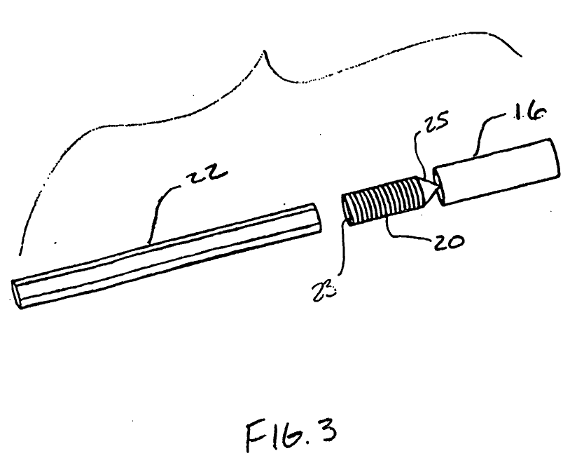

It will be understood that the matrix 16 may be formed of various other biologically compatible materials known in the art. The composition of the matrix 16 may also be configured to contain osteoinductive substances, or any variety of substances known in the art to promote healing. Some embodiments of the matrix 16 may also be configured to resorb or be converted to host bone over time, while other embodiments of the matrix 16 may not be resorbable. Moreover, it will be understood that graft in-growth enhancing factors may be delivered using this technique as well. The graft in-growth enhancing factors may include any variety of materials known to those skilled in the art to improve the development of the graft or otherwise improve healing. As discussed more fully below, the matrix 16 may be used to attach a graft 21, as shown schematically in FIG. 4, to the bone 18. The graft 21 may be a soft tissue graft, such as a ligament or tendon implant, formed in any manner known to those skilled in the art.

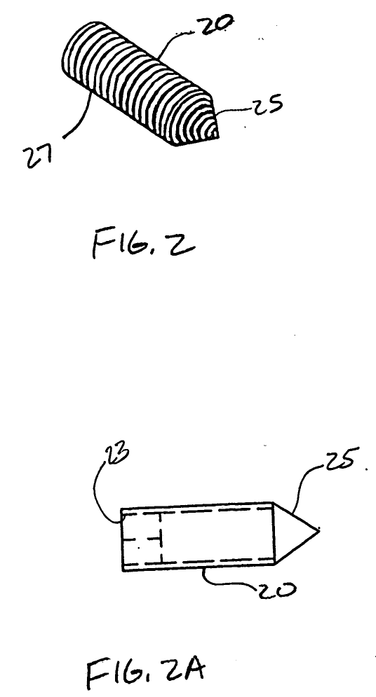

A fastener 20, as shown in FIGS. 2, 2A, 3, 7, and 8 may also be used to attach the graft 21 to the bone 18. The fastener 20 may be formed as a threaded screw, a non-threaded expanding fastener, or any other such device known to those skilled in the art. Moreover, the fastener 20 may be formed of any suitable material known to those skilled in the art. The fastener 20 may be driven by a driver 22, such as a hex driver, or a driver having a hexagonal cross-sectional shape. Accordingly, the fastener 20 may include a fitting 23 for engagement with the driver 22. It will be understood that the fastener 20 and fitting 23 may be formed to be compatible with various different drivers within the scope of the present disclosure, and should in no way be limited to a hex driver. For example, the driver 22 may include any variety of shapes, including polygonal or curved shapes, and the fitting 23 may be formed compatible with the driver 22. Moreover, it will be understood that the fastener 20 may be sized slightly larger in diameter than the insertion device 10 to allow the fastener 20 to apply additional compressive force to the hardened matrix 16 when the fastener 20 is installed in the matrix 16 as discussed more fully below. However, it will be understood that the fastener 20 may be formed in various sizes and configurations, and the fastener 20 is not limited to any particular dimension.

A taper 25 may be provided on a distal end of the fastener 20 to facilitate insertion of the fastener 20 in the matrix 16. Moreover, the fastener 20 may include threads 27 on a portion of, or an entire length of the fastener 20, to facilitate driving the fastener 20 into the matrix.

Installation of the matrix 16 and fastener 20 in the bone 18 will now be described, referring to FIGS. 4-8. As shown in FIG. 4, a tunnel 24 may be formed in the bone 18, such as a tibia. The tunnel 24 may be formed in any manner known to those skilled in the art, such as by drilling or boring, for example. It will be understood that while the principles of the present disclosure are discussed herein with reference directed to use in the tibia, the principles of the present disclosure may also be used in other bones to attach implants in other portions of the body.

The insertion device 10 may be loaded with a matrix 16, and one or more soft tissue graft strands 21 may be located around the shaft of the insertion device 10. The insertion device 10 may be inserted in the tunnel 24 and a gasket or retainer (not shown) may be placed around the insertion device 10, external to the tunnel 24 to restrict flow of liquid material out of the tunnel 24 until the matrix 16 has solidified. Any variety of gasket or retainer mechanism known to those skilled in the art may be used to maintain the matrix 16 within the tunnel 24, or in some instances, the bone may be oriented such that the matrix 16 may be held in place by gravity such that no additional mechanism may be required to maintain the matrix 16 within the tunnel 24. Other embodiments may not require a retaining mechanism due to the stiffness of the matrix 16 even while in a liquid state. One embodiment of the retainer mechanism may be formed of a material that may not adhere to the matrix 16 such that removal of the retainer may be facilitated. Another embodiment may utilize a retainer mechanism that may remain in place attached to the bone.

As shown in FIG. 5, the matrix 16 may be placed in the tunnel 24 by depressing the plunger 12 to force the matrix 16 out of the insertion device 10 through the fenestrations 14. In one embodiment, the insertion device 10 may remain in place within the tunnel 24 until the matrix 16 solidifies as depicted in FIGS. 3 and 6-8. Accordingly, the insertion device 10 may be positioned such that the matrix 16 is placed as desired. Other embodiments may involve removing the insertion device 10 while the matrix 16 is in a liquid state.

As shown in FIG. 6, the insertion device 10 may be removed after the matrix 16 has solidified and the matrix 16 may remain in the tunnel 24. It will be appreciated that when the insertion device 10 is removed, a chamber 28 may remain formed in the matrix 16 for receiving the fastener 20. Accordingly, the insertion device 10 may form a negative mold such that the position of the insertion device 10 in the tunnel 24 while the matrix 16 is in a liquid state may determine the location of the chamber 28. Moreover, the configuration of the chamber 28 may be determined by the configuration of the insertion device 10. It will also be understood, as discussed above, that the insertion device 10 may be removed without creating a chamber 28 in alternative embodiments, within the scope of the present disclosure.

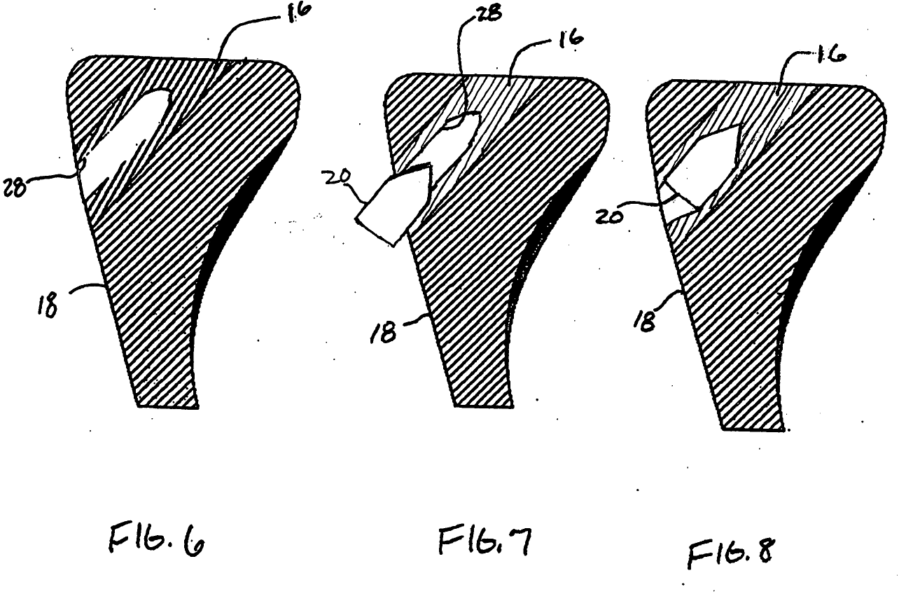

As shown in FIGS. 7-8, the fastener 20 may be placed in the solidified matrix 16 to apply a compressive force within the tunnel 24 to fix the graft to the tunnel 24. In one embodiment, the fastener 20 may be larger than the chamber 28 such that the matrix 16 may be compressed against the tunnel 24 as the fastener 20 is driven into the chamber 28. It will be understood that other varieties of fasteners may also be provided to compress the matrix 16 against the side of tunnel 24. As such, some aspects of the present technique may serve to resolve some of the drawbacks of the prior art methods by substituting a bio-compatible compound that may be introduced in the bone 18 in a liquid state using the insertion device 10 that may also serve as a negative mold for the fastener 20.

It will be understood that the matrix 16 may form an attachment means for placing in a bone in a liquid state and thereafter solidifying. Moreover, it will be appreciated that the structure and apparatus disclosed herein is merely exemplary of an attachment means for placing in a bone in a liquid state and thereafter solidifying, and it should be appreciated that any structure, apparatus or system for attaching which performs functions the same as, or equivalent to, those disclosed herein are intended to fall within the scope of an attachment means for placing in a bone in a liquid state and thereafter solidifying, including those structures, apparatus or systems for placing in a bone in a liquid state and thereafter solidifying which are presently known, or which may become available in the future. Anything which functions the same as, or equivalently to, an attachment means for placing in a bone in a liquid state and thereafter solidifying falls within the scope of this element.

In accordance with the features and combinations described above, a useful method of attaching a ligament or tendon implant to a bone of a patient includes the steps of:
(a) forming a tunnel in the bone;
(b) selecting a matrix that resides in a liquid state and is capable of solidifying;
(c) placing the matrix and the implant into the tunnel while the matrix is in the liquid state; and
(d) allowing the matrix to solidify.

Those having ordinary skill in the relevant art will appreciate the advantages provide by the features of the present disclosure. For example, it is a feature of the present disclosure to provide an apparatus for attaching a ligament or tendon implant to a bone of a patient which is simple in design and manufacture. Another feature of the present disclosure is to provide such an apparatus which may utilize compression bio-compatible materials that may be introduced in a liquid state. It is a further feature of the present disclosure, in accordance with one aspect thereof, to provide a method of attaching a ligament or tendon implant to a bone of a patient which utilizes an all endosteal technique and eliminates metallic artifact on imaging studies. Moreover, the present method may reduce the use of foreign, non-biodegradable, materials in ACL reconstruction.

In the foregoing Detailed Description, various features of the present disclosure are grouped together in a single embodiment for the purpose of streamlining the disclosure. This method of disclosure is not to be interpreted as reflecting an intention that the claimed disclosure requires more features than are expressly recited in each claim. Rather, as the claims will reflect, inventive aspects lie in less than all features of a single foregoing disclosed embodiment.

It is to be understood that the above-described arrangements are only illustrative of the application of the principles of the present disclosure. Numerous modifications and alternative arrangements may be devised by those skilled in the art without departing from the spirit and scope of the present disclosure and the appended claims are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

1. A method of attaching a ligament or tendon implant to a bone of a patient, the method comprising:
(a) forming a tunnel in the bone;
(b) selecting a matrix that resides in a liquid state and is capable of solidifying;
(c) placing the matrix and the implant into the tunnel with an insertion device while the matrix is in the liquid state; and
(d) allowing the matrix to solidify.

2. The method of claim 1, wherein part (c) comprises joining said implant with said insertion device.

3. The method of claim 2, further comprising ejecting said matrix through fenestrations in said insertion device.

4. The method of claim 2, further comprising maintaining said insertion device in said tunnel until said matrix solidifies.

5. The method of claim 4, further comprising removing said insertion device to form a chamber in said matrix.

6. The method of claim 1, further comprising forming a chamber in said matrix.

7. The method of claim 1, wherein selecting said matrix comprises selecting a matrix formed of a calcium-based paste.

8. The method of claim 1, wherein selecting said matrix comprises selecting a matrix formed of a polymer.

9. The method of claim 1, further comprising compressing the matrix against the bone.

10. The method of claim 9, wherein compressing the matrix against the bone comprises installing a fastener in the matrix.

11. The method of claim 9, wherein compressing the matrix against the bone comprises installing a threaded screw in the matrix.

12. The method of claim 1, wherein part (c) comprises placing an insertion device in the tunnel and ejecting said matrix through fenestrations in said insertion device;
wherein the method further comprises maintaining said insertion device in said tunnel until said matrix solidifies;
wherein the method further comprises removing said insertion device to form a chamber in said matrix;
wherein the method further comprises selecting said matrix from one of a group consisting of a calcium-based material, a polymer material and a magnesium material;
wherein the method further comprises installing a fastener in the chamber in the matrix to thereby compress the matrix against the bone;
wherein installing said fastener in the chamber comprises installing a threaded screw in the chamber;
wherein said threaded screw is sized to have a larger diameter than a diameter of said insertion device such that said diameter of said threaded screw is larger than a diameter of said chamber.

13. A method of attaching a ligament or tendon implant to a bone of a patient, the method comprising:
(a) selecting a matrix that resides in a liquid state and is capable of solidifying;
(b) placing the matrix into the bone with an insertion device while the matrix is in the liquid state; and
(c) maintaining the insertion device in the matrix as said matrix solidifies to form a chamber for receiving a fastener in said matrix.

14. The method of claim 13, further comprising placing said implant in said bone with said insertion device.

15. The method of claim 13, further comprising removing said insertion device.

16. The method of claim 15, further comprising installing a fastener in said chamber.

17. The method of claim 13, further comprising compressing the matrix against the bone.

18. The method of claim 13, further comprising forming a tunnel in the bone.

19. A method of attaching a ligament or tendon implant to a bone of a patient, the method comprising:
(a) forming a tunnel in the bone;
(b) selecting a matrix that resides in a liquid state and is capable of solidifying;
(c) placing the matrix and the implant into the tunnel while the matrix is in the liquid state; and
(d) installing a fastener in the matrix to compress the matrix against the bone.

20. The method of claim 19, further comprising allowing the matrix to solidify.

21. The method of claim 19, further comprising selecting said matrix from one of a group consisting of a calcium-based material, a polymer material and a magnesium material.

22. The method of claim 19, wherein part (c) comprises placing an insertion device in the tunnel.

23. The method of claim 22, further comprising maintaining said insertion device in said tunnel until said matrix solidifies and thereafter removing said insertion device.

24. The method of claim 19, further comprising forming a chamber in said matrix.

25. A method of attaching a ligament or tendon implant to a bone of a patient, the method comprising:
(a) forming a tunnel in the bone;
(b) selecting a matrix that resides in a liquid state and is capable of solidifying;
(c) placing the matrix into the tunnel while the matrix is in the liquid state; and
(d) forming a chamber in said matrix for receiving a fastener such that when a fastener is installed in said chamber, a compressive force is applied to attach said implant to said bone.

26. The method of claim 25, wherein part (c) comprises placing an insertion device in the tunnel.

27. The method of claim 26, further comprising maintaining said insertion device in said tunnel until said matrix solidifies to form said chamber.

28. The method of claim 25, further comprising compressing the matrix against the bone.

29. The method of claim 28, wherein compressing the matrix against the bone comprises installing a fastener in the matrix.

30. The method of claim 25, wherein part (b) further comprises selecting said matrix from one of a group consisting of a calcium-based material, a polymer material and a magnesium material.

31. An apparatus for attaching a ligament or tendon implant to a bone of a patient, said apparatus comprising:
a matrix formed in a liquid state for joining with said implant and said bone, said matrix undergoing a reaction to solidify; and
a fastener for placing in said matrix for compressing said matrix and said implant against said bone to thereby attach said implant to said bone.

32. The apparatus of claim 31, wherein said matrix is formed of a calcium-based material.

33. The apparatus of claim 31, wherein said matrix is formed of a polymer material.

34. The apparatus of claim 31, wherein said matrix comprises a material configured to resorb into bone.

35. The apparatus of claim 31, wherein said matrix comprises a material configured to enhance healing.

36. The apparatus of claim 31, wherein said fastener comprises a threaded screw.

37. The apparatus of claim 31, wherein said fastener comprises a fitting for receiving a driver.

38. The apparatus of claim 37, wherein said fitting is configured for receiving a driver having a hexagonal shape.

39. The apparatus of claim 31, wherein said fastener comprises a tapered end.

40. The apparatus of claim 31, further comprising an insertion device for installing said matrix in said bone.

41. The apparatus of claim 40, wherein said insertion device comprises a housing wall defining a reservoir.

42. The apparatus of claim 41, wherein said housing wall comprises one or more fenestrations.

43. The apparatus of claim 40, wherein said insertion device comprises a plunger for forcing the matrix out of the insertion device.

44. The apparatus of claim 40, wherein said fastener has a diameter that is larger than a diameter of said insertion device.

45. An apparatus for attaching a ligament or tendon implant to a bone of a patient, said apparatus comprising:
attachment means for placing in said bone in a liquid state and thereafter solidifying; and
a fastener for placing in said attachment means for compressing said attachment means and said implant against said bone to thereby attach said implant to said bone.

46. The apparatus of claim 45, wherein said attachment means is formed of a calcium-based material.

47. The apparatus of claim 45, wherein said attachment means is formed of a polymer material.

48. The apparatus of claim 45, wherein said fastener comprises a threaded screw.

49. The apparatus of claim 45, further comprising an insertion device for installing said matrix in said bone.

50. The apparatus of claim 49, wherein said insertion device comprises a housing wall defining a reservoir.

51. The apparatus of claim 50, wherein said housing wall comprises one or more fenestrations and a plunger for forcing the matrix out of the insertion device through the one or more fenestrations.

52. The apparatus of claim 49, wherein said fastener has a diameter that is larger than a diameter of said insertion device.

53. The apparatus of claim 45, wherein said attachment means is formed of a magnesium material.

54. The method of claim 1, wherein selecting said matrix comprises selecting a matrix formed of a magnesium material.
